(19) United States
(12) Patent Application Publication
(10) Pub. No.: US 2005/0250714 A1
(43) Pub. Date: Nov. 10, 2005

(54) Crystalline forms of 9-(S)-erythromycylamine

(76) Inventors: Kangwen L. Lin, Fremont, CA (US); Augustus O. Okhamafe, Concord, CA (US); Patricia Frech, San Diego, CA (US)

Correspondence Address: Chiron Corporation;Intellectual Property - R440, P.O. Box 8097, Emeryville, CA, 94662-8097, US

(21) Appl. No.: 11/122,537

(22) Filed: May 5, 2005

Related U.S. Application Data
(60) Provisional application No. 60/568,638, filed on May 6, 2004.
(60) Provisional application No. 60/636,452, filed on Dec. 16, 2004.

(51) Int. Cl. A61K 31/7052; C07H 17/08

(52) U.S. Cl. 514/29; 536/7.4

(57) ABSTRACT

The present invention provides novel crystalline forms of the macrolide antibiotic 9-(S)-erythromycylamine, and methods of preparing and using the same.

### CROSS REFERENCE TO RELATED APPLICATIONS

This applications claims the benefit of U.S. Ser. Nos. 60/568,638, filed May 6, 2004 and 60/636,452, filed Dec. 6, 2004, the disclosures of each of which are incorporated herein by reference in their entireties.

### FIELD OF THE INVENTION

The present invention relates to crystalline forms of the macrolide antibiotic 9-(S)-erythromycylamine, compositions thereof, and methods of their preparation.

### BACKGROUND OF THE INVENTION

9-(S)-Erythromycylamine belongs to the family of macrolide antibiotics and is widely known to have bacteriostatic or bactericidal activity for most gram positive bacteria, in particular Streptococci, and good activity toward respiratory pathogens. Macrolides of this type have been proven safe and effective for many respiratory infections, and are useful in patients with penicillin allergy.

9-(S)-Erythromycylamine can be prepared from Erythromycin by conversion of the 9-oxo moiety in Erythromycin to a 9-amino moiety by well-documented semisynthetic procedures. For example, methods for preparing 9-(S)- and 9-(R)-erythromycylamines are described by Massey et al., Tetrahedron Letters, 157 (1970); Wildsmith, Tetrahedron Letters, 29 (1972); and Massey et al., J. Med Chem., 17 105-107 (1974). The structure of 9-(S)-erythromycylamine is provided below.

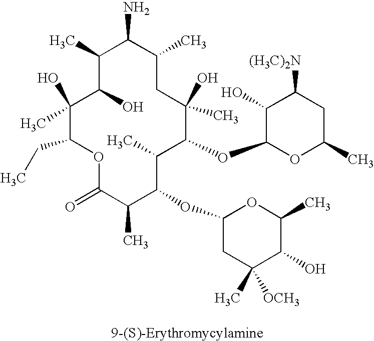

It is well known that the crystalline form of a particular drug is often an important determinant of the drug's ease of preparation, stability, solubility, storage stability, ease of formulation and in vivo pharmacology. Crystalline forms occur where the same composition of matter crystallizes in a different lattice arrangement resulting in different thermodynamic properties and stabilities specific to the particular crystalline form. In deciding which form is preferable, the numerous properties of the forms are compared and the preferred form chosen based on the many physical property variables. It is entirely possible that one form can be preferable in some circumstances where certain aspects such as ease of preparation, stability, etc are deemed to be critical. In other situations, a different form may be preferred for greater solubility and/or superior pharmacokinetics.

Because improved drug formulations, showing, for example, better bioavailability or better stability are consistently sought, there is an ongoing need for new or purer polymorphic forms of existing drug molecules. The crystalline forms of 9-(S)-erythromycylamine described herein help meet these and other needs.

### SUMMARY OF THE INVENTION

The present invention provides a crystalline form of 9-(S)-erythromycylamine which is Form A.

The present invention further provides Form A having a powder X-ray diffraction pattern comprising characteristic peaks, in terms of 2θ, at about 17.7° and about 19.1°.

The present invention further provides Form A wherein the powder X-ray diffraction pattern comprises at least 5 characteristic peaks, in terms of 2θ selected from about 8.8°, about 10.7°, about 11.4°, about 12.9°, about 14.1°, about 15.6°, about 16.2°, about 16.6°, about 17.7, about 19.1°, about 20.3°, about 21.0°, about 21.9°, about 22.4°, about 24.0°, about 24.5°, about 24.8°, and about 26.0°.

The present invention further provides Form A having a powder X-ray diffraction pattern substantially as shown in FIG. 1.

The present invention further provides Form A having a differential scanning calorimetry trace showing an endotherm between about 190 and 200° C.

The present invention further provides Form A having a differential scanning calorimetry trace substantially as shown in FIG. 2.

The present invention further provides a composition comprising Form A.

The present invention further provides a composition comprising Form A wherein at least about 50% by weight of total 9-(S)-erythromycylamine in said composition is present as Form A.

The present invention further provides a composition comprising Form A wherein at least about 70% by weight of total 9-(S)-erythromycylamine in said composition is present as Form A.

The present invention further provides a composition comprising Form A wherein at least about 80% by weight of total 9-(S)-erythromycylamine in said composition is present as Form A.

The present invention further provides a composition comprising Form A wherein at least about 90% by weight of total 9-(S)-erythromycylamine in said composition is present as Form A.

The present invention further provides a composition comprising Form A wherein at least about 95% by weight of total 9-(S)-erythromycylamine in said composition is present as Form A.

The present invention further provides a composition comprising Form A wherein at least about 97% by weight of total 9-(S)-erythromycylamine in said composition is present as Form A.

The present invention further provides a composition comprising Form A wherein at least about 98% by weight of total 9-(S)-erythromycylamine in said composition is present as Form A.

The present invention further provides a composition comprising Form A wherein at least about 99% by weight of total erythromycylamine in said composition is present as Form A.

The present invention further provides a composition comprising Form A and a pharmaceutically acceptable carrier.

The present invention further provides a composition comprising Form A that is suitable for inhalation.

The present invention further provides a composition consisting essentially of 9-(S)-erythromycylamine wherein at least 95% by weight of said 9-(S)-erythromycylamine is present in said composition as Form A.

The present invention further provides a composition consisting essentially of 9-(S)-erythromycylamine wherein at least 97% by weight of said 9-(S)-erythromycylamine is present in said composition as Form A.

The present invention further provides a composition consisting essentially of 9-(S)-erythromycylamine wherein at least 98% by weight of said 9-(S)-erythromycylamine is present in said composition as Form A.

The present invention further provides a composition consisting essentially of 9-(S)-erythromycylamine wherein at least 99% by weight of said 9-(S)-erythromycylamine is present in said composition as the crystalline form of claim 1.

The present invention further provides a method of preparing Form A comprising heating solid 9-(S)-erythromycylamine for a time and under conditions suitable for forming Form A.

The present invention further provides a method of preparing Form A comprising heating solid 9-(S)-erythromycylamine for a time and under conditions suitable for forming Form A wherein said solid 9-(S)-erythromycylamine comprises amorphous 9-(S)-erythromycylamine. The solid 9-(S)-erythromycylamine can be heated to about 100 to about 200° C. The solid 9-(S)-erythromycylamine can be heated to about 170° C. for about 10 to about 30 minutes. The solid 9-(S)-erythromycylamine can be heated to about 170 to about 190° C. for about 5 minutes to about 2 hours.

The present invention further provides a method of preparing Form A comprising heating solid 9-(S)-erythromycylamine for a time and under conditions suitable for forming Form A wherein said solid 9-(S)-erythromycylamine comprises a 9-(S)-erythromycylamine crystalline form other than Form A.

In some embodiments, the solid 9-(S)-erythromycylamine can be heated to about 100 to about 200° C. In some embodiments, the solid 9-(S)-erythromycylamine can be heated to at least about 160° C. for about 10 to about 30 minutes. In some embodiments, the solid 9-(S)-erythromycylamine can be heated to at least about 170° C. for about 10 to about 30 minutes. In some embodiments, the solid 9-(S)-erythromycylamine can be heated to about 160 to about 200° C. for about 5 minutes to about 2 hours. In some embodiments, the solid 9-(S)-erythromycylamine can be heated to about 170 to about 190° C. for about 5 minutes to about 2 hours.

The present invention further provides a method of preparing Form A comprising inducing precipitation of Form A from a high boiling solvent at elevated temperature by addition of an anti-solvent. In some embodiments, the elevated temperature is above about 80° C. and less than about the melting point of said crystalline form. In some embodiments, the elevated temperature is about 80 to about 180° C.

The present invention further provides a method of preparing Form A comprising inducing precipitation of From A from a high boiling solvent at elevated temperature by evaporation of said high boiling solvent. In some embodiments, the elevated temperature is above about 80° C. and less than about the melting point of said crystalline form. In some embodiments, the elevated temperature is about 80 to about 180° C.

The present invention further provides a method of preparing Form A comprising inducing precipitation of Form A from a high boiling solvent at elevated temperature by cooling of said high boiling solvent. In some embodiments, the high boiling solvent is cooled from a high temperature of about 180 to about 200° C. to a reduced temperature of about 80 to about 180° C.

The present invention further provides a crystalline form prepared by any of the above methods.

The present invention further provides a crystalline form of 9-(S)-erythromycylamine which is Form B.

The present invention further provides a crystalline form of 9-(S)-erythromycylamine which is Form B having an XRPD pattern substantially as shown in FIG. 11.

The present invention further provides Form B having a differential scanning calorimetry trace showing a broad endotherm between about 1 10 and about 120° C.

The present invention further provides Form B having a differential scanning calorimetry trace showing a broad endotherm between about 114 and about 118° C. and a broad endotherm between about 295 and about 305° C.

The present invention further provides Form B having a differential scanning calorimetry trace substantially as shown in FIG. 9.

The present invention further provides a composition comprising Form B.

The present invention further provides a composition comprising Form B wherein at least about 50% by weight of total 9-(S)-erythromycylamine in said composition is present as Form B.

The present invention further provides a method of preparing the Form B comprising suspending solid 9-(S)-erythromycylamine in an aqueous solvent for a time and under conditions suitable for forming Form B. In some embodiments, the aqueous solvent is heated to about 50 to about 200° C.

The present invention further provides a method of preparing Form B comprising inducing precipitation of Form B from a solution by cooling or evaporation of said solution. In some embodiments, the method further comprises adding isolates of substantially pure Form B to said solution.

The present invention further provides a crystalline form of 9-(S)-erythromycylamine which is Form C.

The present invention further provides Form C having a differential scanning calorimetry trace showing a broad endotherm between about 70 and about 80° C.

The present invention further provides Form C having a differential scanning calorimetry trace showing a broad endotherm between about 70 and about 80° C. and a small endotherm between about 110 and about 115° C.

The present invention further provides Form C having a differential scanning calorimetry trace substantially as shown in FIG. 3.

The present invention further provides a composition Form C. In some embodiments, at least about 50% by weight of total 9-(S)-erythromycylamine in said composition is present as Form C.

The present invention further provides a method of preparing Form C comprising suspending solid 9-(S)-erythromycylamine in a solution comprising ethanol, isopropanol, acetonitrile/isopropanol, or methanol/isopropanol or mixtures thereof for a time and under conditions suitable for forming Form C.

The present invention further provides a method of preparing Form C comprising inducing precipitation of Form C from a solution comprising ethanol, isopropanol, acetonitrile/isopropanol, or methanol/isopropanol or mixtures thereof by cooling or evaporation of said solution. In some embodiments, the method further comprises adding isolates of substantially pure Form C to said solution.

The present invention further provides a composition comprising Form A and Form C.

The present invention further provides a crystalline form of 9-(S)-erythromycylamine which is Form D.

The present invention further provides Form D having a differential scanning calorimetry trace showing a broad endotherm between about 75 and about 85° C. and two sharp endotherms at about 105° C. and 110° C.

The present invention further provides Form D having a differential scanning calorimetry trace showing a broad endotherm between about 75 and about 85° C., two sharp endotherms at about 105° C. and about 110° C. and a small exotherm between about 180 and about 190° C.

The present invention further provides Form D having a differential scanning calorimetry trace substantially as shown in FIG. 4.

The present invention further provides a composition comprising Form D. In some embodiments, at least about 50% by weight of total 9-(S)-erythromycylamine in said composition is present as Form D.

The present invention further provides a method of preparing Form D comprising suspending solid 9-(S)-erythromycylamine in a solvent comprising isopropanol, dichloromethane/isopropanol, or toluene/isopropanol or mixtures thereof for a time and under conditions suitable for forming Form D.

The present invention further provides a method of preparing Form D comprising inducing precipitation of Form D from a solution comprising isopropanol, dichloromethane/isopropanol, or toluene/isopropanol or mixtures thereof by cooling or evaporation of said solution. In some embodiments, the method further comprises adding isolates of substantially pure Form D to said solution.

The present invention further provides Form D having an X-ray powder diffraction pattern comprises at least 3 characteristic peaks, in terms of 2θ, selected from about 5.0°, about 5.6°, about 11.1°, about 14.9°, and about 17.7°.

The present invention further provides a crystalline form of 9-(S)-erythromycylamine which is Form E.

The present invention further provides Form E having a differential scanning calorimetry trace showing a broad endotherm between about 62 and about 72° C. and an endotherm between about 100 and about 110° C.

The present invention further provides Form E having a differential scanning calorimetry trace substantially as shown in FIG. 5.

The present invention further provides a composition comprising Form E. In some embodiments, at least about 50% by weight of total 9-(S)-erythromycylamine in said composition is present as Form E. In some embodiments, at least about 95% by weight of total 9-(S)-erythromycylamine in said composition is present as Form E.

The present invention further provides a method of preparing Form E comprising suspending solid 9-(S)-erythromycylamine in a solvent comprising acetonitrile/isopropanol or methanol/isopropanol or mixtures theeof for a time and under conditions suitable for forming Form E.

The present invention further provides a method of preparing Form E comprising inducing precipitation of Form E from a solvent comprising acetonitrile/isopropanol or methanol/isopropanol or mixtures thereof by cooling or evaporation of said solution. In some embodiments, the method further comprises adding isolates of substantially pure Form E to said solution.

The present invention further provides Form E having a powder X-ray diffraction pattern comprising at least 3 characteristic peaks, in terms of 2θ, selected from about 5.6°, about 10.1°, about 10.9°, about 11.1°, about 17.5°, and about 17.7°.

The present invention further provides a crystalline form of 9-(S)-erythromycylamine which is Form F.

The present invention further provides Form F having a powder X-ray diffraction pattern comprising a prominent peak, in terms of 2θ, at about 11.5°.

The present invention further provides From F having a powder X-ray diffraction pattern comprising characteristic peaks, in terms of 2θ, at about 5.5°, about 7.0°, and about 11.5°.

The present invention further provides a composition comprising Form F. In some embodiments, at least about 40% by weight of total 9-(S)-erythromycylamine in said composition is present as Form F. In some embodiments, at least about 95% by weight of total 9-(S)-erythromycylamine in said composition is present as Form F.

The present invention further provides a method of preparing Form F comprising suspending solid 9-(S)-erythromycylamine in a solvent comprising ethanol or acetone/hexanes or mixtures thereof for a time and under conditions suitable for forming Form F.

The present invention further provides a method of preparing Form F comprising inducing precipitation of Form F from a solution comprising ethanol or acetone/hexanes or mixtures thereof by cooling or evaporation of said solution. In some embodiments, the method further comprises adding isolates of substantially pure Form F to said solution.

The present invention further provides a crystalline form of 9-(S)-erythromycylamine which is Form G.

The present invention further provides Form G having a differential scanning calorimetry trace showing an endotherm between about 100 and about 110° C.

The present invention further provides Form G having a differential scanning calorimetry trace substantially as shown in FIG. 6.

The present invention further provides a composition comprising Form G wherein at least about 50% by weight of total 9-(S)-erythromycylamine in said composition is present as Form G. In some embodiments, at least about 90% by weight of total 9-(S)-erythromycylamine in said composition is present as Form G. In some embodiments, at least about 95% by weight of total 9-(S)-erythromycylamine in said composition is present as Form G.

The present invention further provides a method of preparing Form G comprising suspending solid 9-(S)-erythromycylamine in a solution comprising acetonitrile for a time and under conditions suitable for forming Form G.

The present invention further provides a method of preparing Form G comprising inducing precipitation of Form G from a solution comprising acetonitrile by evaporation of said solution. In some embodiments, the method further comprises adding isolates of substantially pure Form G to said solution.

The present invention further provides Form G having a powder X-ray diffraction pattern with a prominent peak, in terms of 2θ, at about 9.3°.

The present invention further provides a crystalline form of 9-(S)-erythromycylamine which is Form H.

The present invention further provides Form H having a differential scanning calorimetry trace showing an endotherm between about 100 and about 110° C.

The present invention further provides Form H having a differential scanning calorimetry trace substantially as shown in FIG. 7.

The present invention further provides a composition comprising Form H wherein at least about 50% by weight of total 9-(S)-erythromycylamine in said composition is present as Form H.

The present invention further provides a composition comprising Form H wherein at least about 90% by weight of total 9-(S)-erythromycylamine in said composition is present as Form H. In some embodiment, at least about 95% by weight of total 9-(S)-erythromycylamine in said composition is present as Form H.

The present invention further provides a method of preparing Form H comprising suspending solid 9-(S)-erythromycylamine in a solution comprising acetonitrile under conditions suitable for forming Form H.

The present invention further provides a method of Form H comprising inducing precipitation of Form H from said solution by cooling said solution. In some embodiments, the method comprises adding isolates of substantially pure Form H to said solution.

The present invention further provides Form H having a powder X-ray diffraction pattern with a prominent peak, in terms of 2θ, at about 9.5°.

The present invention further provides a crystalline form of 9-(S)-erythromycylamine which is Form I.

The present invention further provides Form I having a powder X-ray diffraction pattern comprising characteristic peaks, in terms of 2θ, at about 8.5° and 10.6°.

The present invention further provides Form I having a powder X-ray diffraction pattern comprising at least 5 characteristic peaks, in terms of 2θ, selected from the group consisting of about 8.5°, about 10.6°, about 10.9°, about 17.8°, about 19.4°, and about 21.8°.

The present invention further provides a composition comprising Form I wherein at least about 50% by weight of total 9-(S)-erythromycylamine in said composition is present as Form I. In some embodiments, at least about 90% by weight of total 9-(S)-erythromycylamine in said composition is present as Form I. In some embodiments, at least about 95% by weight of total 9-(S)-erythromycylamine in said composition is present as Form I.

The present invention further provides a method of preparing Form I comprising suspending solid 9-(S)-erythromycylamine in a solution comprising methanol/acetonitrile for a time and under conditions suitable for forming Form I.

The present invention further provides a method of preparing Form I comprising inducing precipitation of Form I from a solution comprising ethanol or methanol/acetonitrile or mixtures thereof by crash precipitation. In some embodiments, the method further comprises adding isolates of substantially pure Form I to said solution.

The present invention further provides a pharmaceutical composition comprising Form I and Form A.

The present invention further provides a crystalline form of 9-(S)-erythromycylamine which is Form J.

The present invention further provides Form J having a powder X-ray diffraction pattern comprising characteristic peaks, in terms of 2θ, at about 17.1° and 21.8°.

The present invention further provides Form J having a powder X-ray diffraction pattern comprising characteristic peaks, in terms of 2θ, at about 17.1°, about 19.5°, about 21.8°, about 22.6°, about 23.1° and about 23.5°.

The present invention further provides Form J having a differential scanning calorimetry trace substantially as shown in FIG. 8.

The present invention further provides a composition comprising Form J wherein at least about 50% by weight of total 9-(S)-erythromycylamine in said composition is present as Form J. In some embodiments, at least about 90% by weight of total 9-(S)-erythromycylamine in said composition is present as Form J.

The present invention further provides a method of preparing Form J comprising suspending solid 9-(S)-erythromycylamine in a solution comprising water/isopropanol for a time and under conditions suitable for forming Form J. In some embodiments, the solution is heated to about 40°C.

The present invention further provides a method of preparing Form J comprising inducing precipitation of Form J from a solution comprising water/isopropanol by cooling or evaporation of said solution. In some embodiments, the method further comprises adding isolates of substantially pure Form J to said solution.

The present invention further provides a composition comprising at least two crystalline forms of 9-(S)-erythromycylamine selected from the group consisting of Form A, Form B, Form C, Form D, Form E, Form F, Form G, Form H, Form I, and Form J.

The present invention further provides Form C having a having a powder X-ray diffraction pattern substantially as shown in FIG. 10.

The present invention further provides Form D having a having a powder X-ray diffraction pattern substantially as shown in FIG. 10.

The present invention further provides Form E having a having a powder X-ray diffraction pattern substantially as shown in FIG. 10.

The present invention further provides Form F having a having a powder X-ray diffraction pattern substantially as shown in FIG. 10.

The present invention further provides Form G having a having a powder X-ray diffraction pattern substantially as shown in FIG. 10.

The present invention further provides Form H having a having a powder X-ray diffraction pattern substantially as shown in FIG. 10.

The present invention further provides Form I having a having a powder X-ray diffraction pattern substantially as shown in FIG. 10.

The present invention further provides a method of treating a bacterial or protozoal infection in a patient comprising administering to said patient a therapeutically effective amount of any one or more of the crystalline forms or compositions thereof provided herein. The present invention further provides a method of treating a bacterial or protozoal infection in a patient comprising administering to said patient a therapeutically effective amount of 9-(S)-erythromycylamine comprising any one or more of the crystalline forms or compositions thereof provided herein. In some embodiments, the crystalline form is Form A. In some embodiments, the bacterial or protozoal infection is a respiratory infection such as severe chronic bronchitis.

### BRIEF DESCRIPTION OF THE DRAWINGS

FIG. 1 shows an X-ray powder diffraction (XRPD) pattern consistent with crystalline Form A.

FIG. 2 shows a differential scanning calorimetry (DSC) thermogram having a sharp endotherm at about 192° C. that is consistent with crystalline Form A.

FIG. 3 shows a differential scanning calorimetry thermogram having a broad endotherm at about 74° C. followed by a small endotherm at 113° C. that is consistent with the crystalline Form C of the invention.

FIG. 4 shows a differential scanning calorimetry thermogram having a broad endotherm at about 80° C. followed by two sharper endotherms at 105° C. and 110° C., that is consistent with the crystalline Form D of the invention.

FIG. 5 shows a differential scanning calorimetry thermogram having a broad endotherm at about 67° C. followed by another endotherm at 105° C. that is consistent with the crystalline Form E of the invention.

FIG. 6 shows a differential scanning calorimetry thermogram having a sharp endotherm at about 106° C. that is consistent with the crystalline Form G of the invention.

FIG. 7 shows a differential scanning calorimetry thermogram having a sharp endotherm at about 106° C. that is consistent with the crystalline Form H of the invention.

FIG. 8 shows an X-ray powder diffraction pattern having peaks at about 17.1°, about 19.5°, about 21.8°, about 22.6°, about 23.1° and about 23.5°, which are consistent with the crystalline Form J of the invention.

FIG. 9 shows a differential scanning calorimetry thermogram having a sharp endotherm at about 116° C. that is consistent with the crystalline Form B of the invention.

FIG. 10 shows seven X-ray powder diffraction patterns consistent with Forms C, D, E, F, G, H, and I.

FIG. 11 shows X-ray powder diffraction patterns consistent with amorphous material (upper), Form A plus amorphous material (middle), and Form B plus amorphous material (lower).

### DETAILED DESCRIPTION

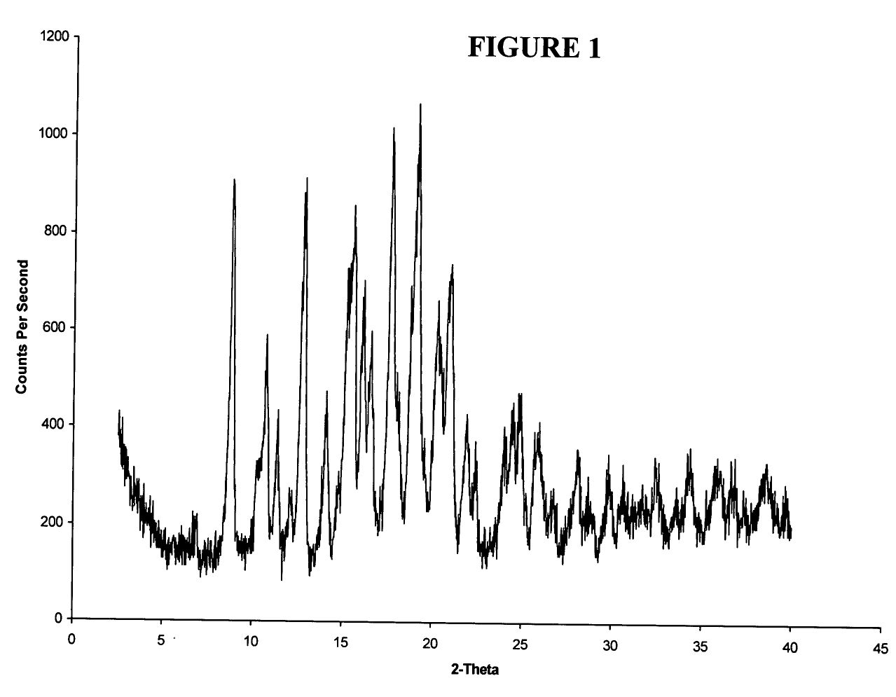

The present invention provides an anhydrous, non-solvated crystalline form of the macrolide antibiotic 9-(S)-erythromycylamine, referred to herein as Form A, which can be identified by one or more solid state analytical methods. The term “crystalline form” refers to crystalline polymorphs of 9-(S)-erythromycylamine including anhydrous forms as well as solvates, and hydrates. Form A can be identified by its powder X-ray diffraction (XRPD) pattern which is provided in FIG. 1. In some embodiments, the crystalline form of the invention has an XRPD pattern substantially as shown in FIG. 1, where the term “substantially” in this instance indicates that 2-theta values for individual peaks can vary about ±0.2° and intensities for individual peaks can vary about ±50 CPS. Powder X-ray diffraction data consistent with Form A is provided in Table 1 below. The relative intensities of the peaks can vary, depending upon the sample preparation technique, the sample mounting procedure and the particular instrument employed. Moreover, instrument variation and other factors can affect the 2-theta values. Therefore, the peak assignments can vary by plus or minus about 0.2°.

TABLE 1

| Observed Peak, | Intensity |
|---|---|
| 2θ (°) | (CPS) |
| 8.8 | 910 |
| 10.7 | 520 |
| 11.4 | 440 |
| 12.9 | 920 |
| 14.1 | 480 |
| 15.6 | 860 |
| 16.2 | 710 |
| 16.6 | 600 |
| 17.7 | 1020 |
| 19.1 | 1070 |
| 20.3 | 670 |
| 21.0 | 740 |
| 21.9 | 430 |
| 22.4 | 380 |
| 24.0 | 410 |
| 24.5 | 460 |
| 24.8 | 480 |
| 26.0 | 420 |

The crystalline Form A of the invention can be recognized by an XRPD pattern having one or more of the peaks listed in Table 1. In some embodiments, the XRPD pattern displays two, three, four, five or more of the peaks listed in Table 1.

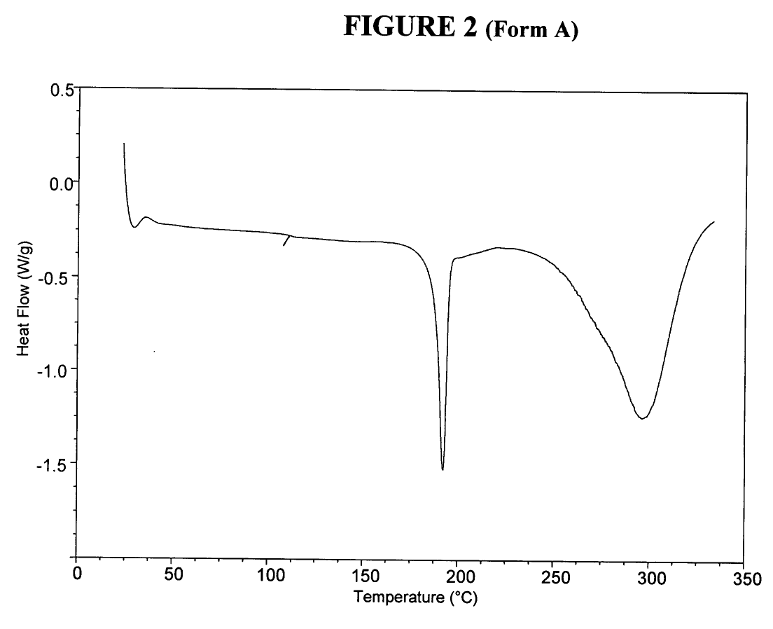

The crystalline Form A of the invention can be further recognized by its differential scanning calorimetry (DSC) which has a characteristic melting endotherm at about 190 to about 200° C. In some embodiments, the characteristic melting endotherm occurs at about 192° C. A typical DSC thermogram for a sample containing substantially pure Form A is provided in FIG. 2. In some embodiments, the crystalline form of the invention has a DSC trace substantially as shown in FIG. 2, where the term “substantially” in this instance indicates that features such as endotherms, exotherms, baseline shifts, etc. can vary about ±4° C. For DSC, it is known that the temperatures observed will depend upon the rate of temperature change as well as sample preparation technique and the particular instrument employed. Thus, the values reported herein relating to DSC thermograms can vary by plus or minus about 4° C.

Form A is also characterized as an anhydrous, non-solvated crystalline material as evidenced by thermogravimetric analysis provided in Example 4 below.

Sorption/desorption data, such as provided below in Example 5, further indicate that Form A can be characterized as a weakly hygroscopic material.

As evidenced herein, Form A has discernable physical and spectroscopic characteristics that distinguish it from amorphous material or other crystalline forms. The physical and spectroscopic characteristics further suggest that Form A is a thermodynamically stable crystalline form. Amorphous preparations of 9-(S)-erythromycylamine that are exposed to high temperature convert to Form A. Further, other crystalline forms of 9-(S)-erythromycylamine have been shown to convert to Form A at high temperature.

The thermodynamic stability of Form A has numerous advantages in the pharmaceutical and other chemical arts. For example, preparations and formulations containing Form A can have longer shelf-lives with increased resistance to humidity and high temperatures compared with compositions containing amorphous, solvated, hydrated or other crystalline forms. Thermodynamic stability can also facilitate the manufacturing of substantially pure Form A for use as an active pharmaceutical ingredient (API).

Form A can be prepared by any appropriate method that allows formation of the desired crystalline form. In some embodiments, Form A can be prepared from solid 9-(S)-erythromycylamine containing, for example, amorphous material or other crystalline forms, including hydrates and solvates. In other embodiments, Form A can be prepared by re-crystallization from high boiling solvents by evaporation or anti-solvent methods. Formation and quantitation of Form A product can be monitored by XRPD, DSC or any other appropriate technique.

Solid state preparations can be carried out, for example, by heating solid 9-(S)-erythromycylamine for a time and under conditions that allows the formation of Form A. For example, the solid 9-(S)-erythromycylamine can be heated to a temperature above the glass transition temperature until Form A is produced. In some embodiments, the temperature can be raised to a temperature of about 100 to about 200° C. In further embodiments, the temperature can be raised to about 100, about 110, about 120, about 130, about 140, about 150, about 160, about 170, about 180, about 190 or about 200° C. The elevated temperature can be held for any length of time sufficient to produce Form A. In some embodiments, the elevated temperature can be held for about 5 minutes, about 10 minutes, about 15 minutes, about 20 minutes, or about 0.5, about 1, about 2, about 5, about 12, about 24, about 36, about 48 or more hours. In some embodiments, the solid 9-(S)-erythromycylamine can be heated to at least about 130° C. for an appropriate amount of time. In some embodiments, the solid 9-(S)-erythromycylamine can be heated to at least about 160° C. for about 10 to about 30 minutes. In some embodiments, the solid 9-(S)-erythromycylamine can be heated to at least about 170° C. for about 10 to about 30 minutes. In further embodiments, the solid 9-(S)-erythromycylamine can be heated to about 160 to about 200° or about 170 to about 190° C. for about 5 minutes to about 2 hours.

In some embodiments, Form A can be prepared by heating a sample containing solid 9-(S)-erythromycylamine, where at least some of the 9-(S)-erythromycylamine of the sample is present in a form other than Form A, to a temperature of about 150° C. for about 10 to bout 30 minutes. In another embodiment, Form A can be prepared by heating a sample containing solid 9-(S)-erythromycylamine, where at least some of the 9-(S)-erythromycylamine of the sample is present in a form other than Form A, to a temperature of about 130 to about 140° C. (e.g., about 134° C.) for about 0.5 to 2 hours. The sample can contain amorphous 9-(S)-erythromycylamine, other crystalline forms, solvates, hydrates, etc., and mixtures thereof. The sample can also contain Form A. Thus, the solid state processes described herein can include increasing the proportion of Form A in a sample of 9-(S)-erythromycylamine containing a mixture of Form A and one or more other forms of 9-(S)-erythromycylamine. In some embodiments, samples can contain primarily amorphous 9-(S)-erythromycylamine, primarily a mixture of amorphous 9-(S)-erythromycylamine and Form A, primarily a crystalline form other than Form A, or primarily a mixture of amorphous 9-(S)-erythromycylamine and a crystalline form other than Form A.

Preparation of Form A from solution can be carried out, for example, by dissolving 9-(S)-erythromycylamine in a high boiling solvent and inducing crystallization of Form A at elevated temperature, such as at or near the solvent boiling temperature, by the addition of an anti-solvent. Suitable high boiling solvents include any solvent that can at least partially dissolve 9-(S)-erythromycylamine and has, for example, a boiling point above about 80, above about 90, above about 100, above about 110, above about 120, above about 130, above about 140, above about 150, about above 160 or above about 170, above about 180, above about 190, or above about 200° C. Some example high boiling solvents include oils, glycols (e.g., ethylene glycol, propylene glycol, etc.), glycerin, propylene carbonate, isopropyl palmitate, ethyl oleate, diethylphthalate, super critical fluids, mixtures thereof and the like. Suitable anti-solvents include any solvent in which 9-(S)-erythromycylamine is not substantially soluble and is at least partially miscible with the high boiling solvent. Suitable anti-solvents can also be characterized as weakly polar or non-polar and have a high boiling point such as a boiling point that is about the same or higher than the high boiling solvent. Some example anti-solvents include benzene, toluene, mixtures thereof and the like. Temperatures at which crystallization can be induced by addition of anti-solvent include the boiling point temperature of the high boiling solvent or any temperature above about 80° C. and less than about the melting point of Form A (e.g., about 80 to about 180° C.).

Preparation of Form A from solution can also be carried out, for example, by dissolving 9-(S)-erythromycylamine in a high boiling solvent and using an evaporation method to produce a supersaturated solution at elevated temperature from which Form A precipitates. Suitable high boiling solvents for the evaporation method include any of those already listed above. Evaporation can be carried out by any appropriate means, including evaporation under reduced pressure or by exposure to a gas stream (e.g., air or nitrogen). The temperatures at which a Form A is precipitated from the supersaturated solution can be about 80 to about 180° C.

Preparation of Form A from solution can also be carried out, for example, by dissolving 9-(S)-erythromycylamine in a high boiling solvent and using a cooling method to produce a supersaturated solution at elevated temperature from which Form A precipitates. Suitable high boiling solvents for the cooling method includes any of those already listed above. The method involves heating the high boiling solvent in which Form A is dissolved to a temperature corresponding to about the melting point of 9-(S)-erythromycylamine (such as about 180 to about 200° C.) and then cooling to a temperature of about 80 to about 180° C.

Methods for preparing Form A can also include the seeding of a solution with seed crystals of Form A obtained from a previous preparation.

The methods for preparation of Form A provided herein can result in substantially pure Form A (e.g., compositions containing less than about 20%, about 10%, about 5%, or about 3% by weight of impurities, amorphous material and/or other crystalline forms) as well as mixtures enriched in Form A (e.g., mixtures containing greater than about 50% by weight Form A relative to, for example, impurities, amorphous material or other crystalline forms). Accordingly, the present invention further provides compositions containing Form A. In some embodiments, at least about 50%, at least about 70%, at least about 80%, at least about 90%, at least about 95%, at least about 97%, at least about 98%, or at least about 99% by weight of total 9-(S)-erythromycylamine in a composition is present as Form A. In further embodiments, compositions of the present invention consist essentially of 9-(S)-erythromycylamine where at least about 95%, at least about 97%, at least about 98%, or at least about 99% of the 9-(S)-erythromycylamine is present in the composition as Form A. In further embodiments, compositions of the present invention consist essentially of 9-(S)-erythromycylamine where at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, of the 9-(S)-erythromycylamine is present in the composition as Form A. In some embodiments, the remainder 9-(S)-erythromycylamine is present as amorphous 9-(S)-erythromycylamine or one or more other crystalline forms (including solvates and hydrates). Amounts of different crystalline forms of 9-(S)-erythromycylamine in a composition can be determined by routine spectroscopic methods, such as X-ray powder diffraction or DSC.

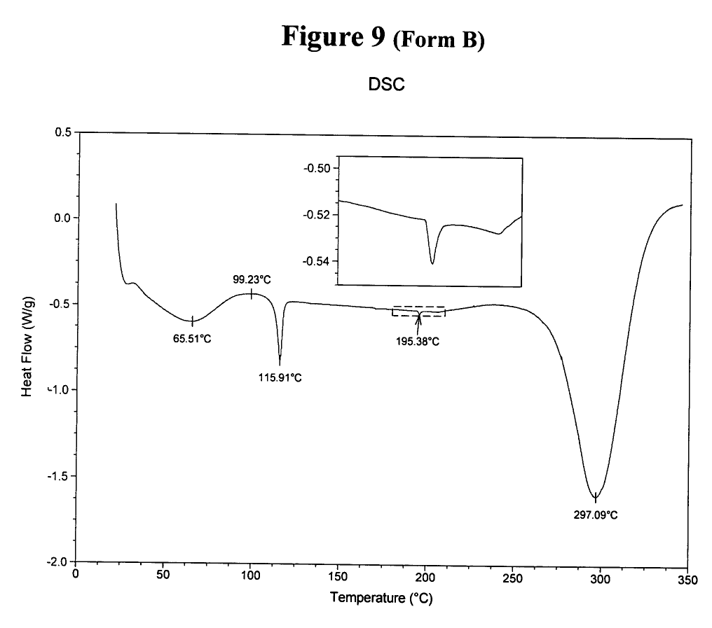

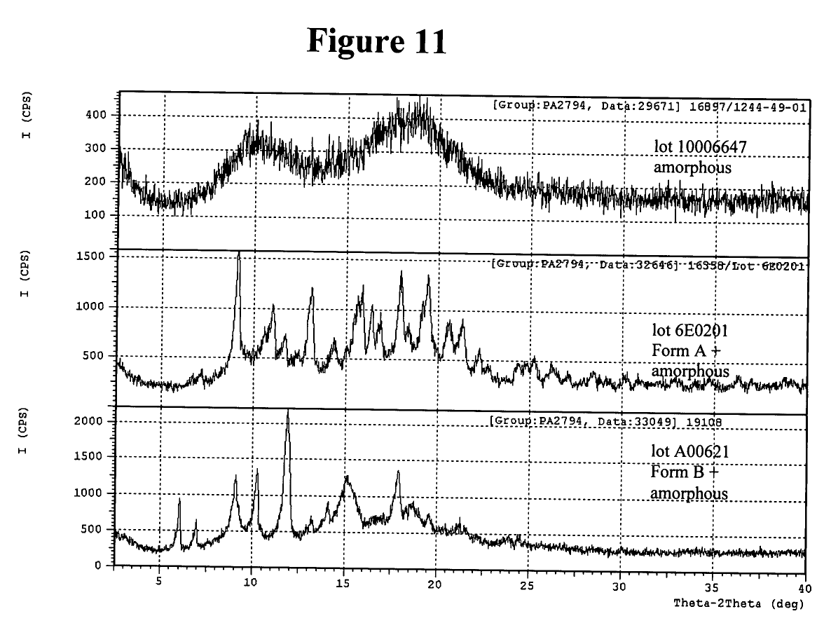

The invention further provides a crystalline form of 9-(S)-erythromycylamine designated Form B. Embodiments of the invention provide Form B having a differential scanning calorimetry trace showing a broad endotherm between about 110 and about 120° C. Further embodiments of the invention provide Form B having a differential scanning calorimetry trace showing a broad endotherm between about 114 and about 118° C. and a broad endotherm between about 295 and about 305° C. Further embodiments of the invention provide Form B having a differential scanning calorimetry trace substantially as shown in FIG. 9. Further embodiments of the invention provide Form B having an XRPD pattern substantially as shown in FIG. 11 (lower).

In some embodiments, the invention provides a composition having at least about 50% by weight of total 9-(S)-erythromycylamine which is present as Form B. Further embodiments provide a composition having at least about 90% by weight of total 9-(S)-erythromycylamine in the composition present as Form B. Further embodiments of the invention provide a composition having at least about 95% by weight of total 9-(S)-erythromycylamine in the composition present as Form B.

The invention further provides a method of preparing Form B comprising dissolving and/or suspending solid 9-(S)-erythromycylamine in an aqueous solution under conditions suitable for forming the crystalline form. The method of preparing Form B can include heating the solution to a temperature of about 50 to about 200° C. In further embodiments, the invention provides methods of preparation of Form B include inducing precipitation of the crystalline form from solution by cooling or evaporation. The method of preparation of Form B can further include inducing precipitation of the crystalline from solution by adding isolates of substantially pure Form B to the solution.

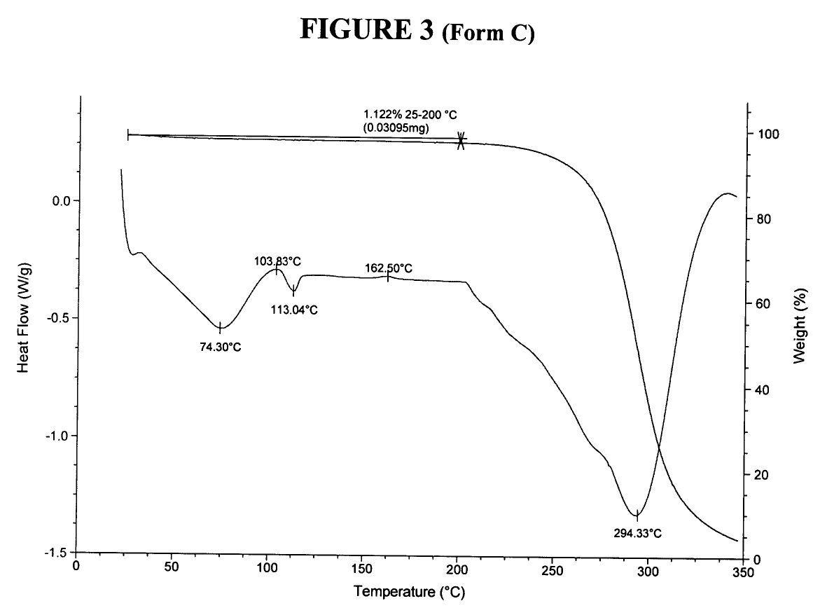

The invention further provides a crystalline form of 9-(S)-erythromycylamine designated Form C. In some embodiments, Form C is characterized as having a differential scanning calorimetry trace showing a broad endotherm between about 70 and about 80° C. In further embodiments, Form C has a differential scanning calorimetry trace showing a broad endotherm between about 70 and about 80° C. and a small endotherm between about 110 and about 115° C. In further embodiments, Form C has a differential scanning calorimetry trace substantially as shown in FIG. 3.

The invention further provides a composition comprising at least about 50% Form C by weight of total 9-(S)-erythromycylamine in the composition. In further embodiments the invention provides a composition comprising at least about 90% by weight Form C of total 9-(S)-erythromycylamine in the composition.

The present invention further provides a method of preparing Form C comprising dissolving and/or suspending solid 9-(S)-erythromycylamine in a solution containing, for example, ethanol, isopropanol, acetonitrile/isopropanol, or methanol/isopropanol or mixtures thereof for a time and under conditions suitable for forming Form C. In some embodiments, the method of preparation of Form C includes inducing precipitation of Form C from a solution containing, for example, ethanol, isopropanol, acetonitrile/isopropanol, or methanol/isopropanol or mixtures thereof by cooling or evaporation of the solution. In further embodiments, the invention provides methods of preparation of Form C by inducing precipitation of Form C from a solution containing, for example, ethanol, isopropanol, acetonitrile/isopropanol, or methanol/isopropanol or mixtures thereof by adding isolates of substantially pure Form C to the solution. In further embodiments, compositions containing Form A and Form C for, for example, treatment of a bacterial infection are provided.

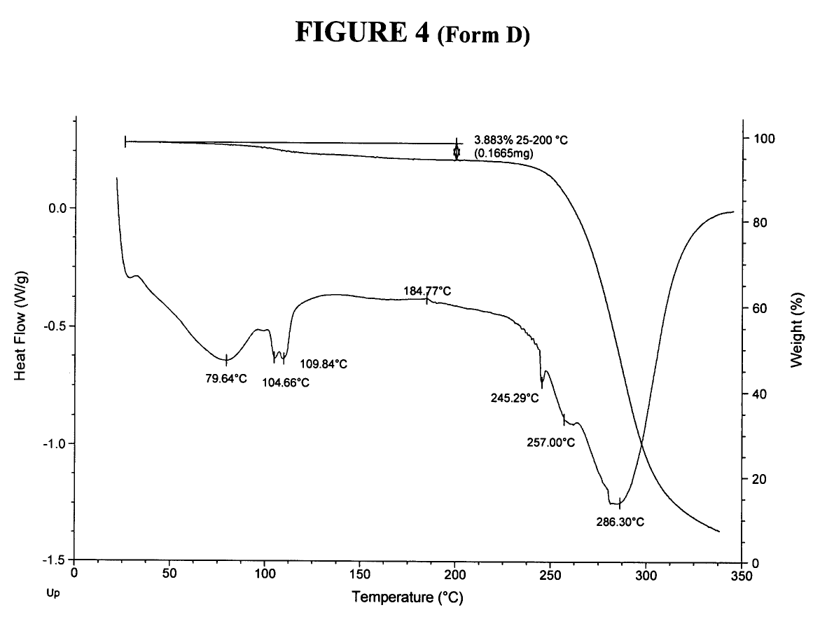

In another aspect of the invention, a crystalline form of 9-(S)-erythromycylamine designated Form D is provided. In some embodiments, Form D has a differential scanning calorimetry trace showing a broad endotherm between about 75 and about 85° C. and two sharp endotherms at about 105° C. and 110° C. In further embodiments, Form D has a differential scanning calorimetry trace showing a broad endotherm between about 75 and about 85° C., two sharp endotherms at about 105° C. and about 110° C. and a small exotherm between about 180 and about 190° C. In further embodiments, Form D has a differential scanning calorimetry trace substantially as shown in FIG. 4. A more particular embodiment of the invention provides a composition having a powder X-ray diffraction pattern with at least 3 characteristic peaks, in terms of 2θ, selected from the group consisting of about 5.0°, about 5.6°, about 11.1°, about 14.9°, and about 17.7°.

The present invention further provides a composition wherein at least about 50% by weight of total 9-(S)-erythromycylamine in said composition is present as Form D. The present invention further provides a composition wherein at least about 90% by weight of total 9-(S)-erythromycylamine in said composition is present as Form D.

In further embodiments, the present invention provides a method of preparing Form D by dissolving and/or suspending solid 9-(S)-erythromycylamine in a solution containing, for example, isopropanol, dichloromethane/isopropanol, or toluene/isopropanol or mixtures thereof for a time and under conditions suitable for forming Form D. Further embodiments provide a method of preparation of Form D by inducing precipitation of Form D from a solution containing, for example, isopropanol, dichloromethane/isopropanol, or toluene/isopropanol or mixtures thereof by cooling or evaporation of the solution. Further embodiments provide a method of preparation of Form D by inducing precipitation of Form D from a solution containing, for example, ethanol, isopropanol, acetonitrile/isopropanol, or methanol/isopropanol or mixtures thereof by adding isolates of substantially pure Form D to the solution.

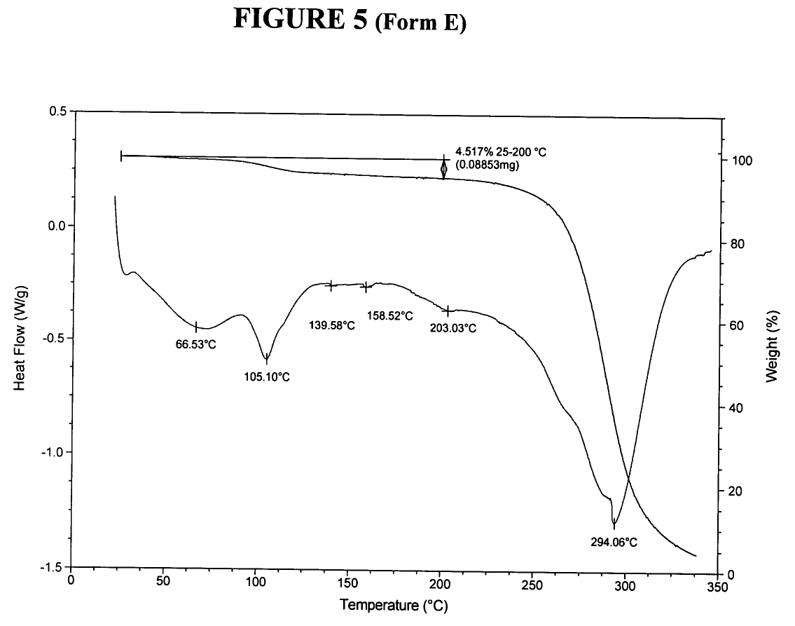

The invention further provides a crystalline form of 9-(S)-erythromycylamine designated Form E. The present invention further provides Form E having a differential scanning calorimetry trace showing a broad endotherm between about 62 and about 72° C. and an endotherm between about 100 and about 110° C. The present invention further provides Form E having a differential scanning calorimetry trace substantially as shown in FIG. 5.

In further embodiments, the invention provides a composition having at least about 50% by weight of total 9-(S)-erythromycylamine in the composition present as Form E. The invention further provides a composition having at least about 90% by weight of total 9-(S)-erythromycylamine in the composition present as Form E. The present invention further provides a composition having at least about 95% by weight of total 9-(S)-erythromycylamine in the composition present as Form E.

The invention further provides a method of preparing Form E comprising dissolving and/or suspending solid 9-(S)-erythromycylamine in a solution containing a suitable solvent such as, for example, acetonitrile/isopropanol or methanol/isopropanol or mixtures thereof for a time and under conditions suitable for forming Form E. The invention further provides a method of preparing Form E by inducing precipitation of Form E from a solution containing, for example, acetonitrile/isopropanol or methanol/isopropanol or mixtures thereof by cooling or evaporation of the solution. The invention further provides a method of preparing Form E by inducing precipitation of Form E from a solution containing, for example, acetonitrile/isopropanol, or methanol/isopropanol or mixtures thereof by adding isolates of substantially pure Form E to the solution.

In some embodiments, the invention provides Form E having a powder X-ray diffraction pattern with at least 3, 4 or 5 characteristic peaks, in terms of 2θ, selected from about 5.6°, about 10.1°, about 10.9°, about 11.1°, about 17.5°, and about 17.7°.

The invention further provides a crystalline form of 9-(S)-erythromycylamine designated Form F. The present invention provides Form F having a powder X-ray diffraction pattern comprising a prominent peak, in terms of 2θ, at about 11.5°. In some embodiments, Form F has a powder X-ray diffraction pattern comprising characteristic peaks, in terms of 2θ, at about 5.5°, about 7.0°, and about 11.5°. In further embodiments, Form F has a differential scanning calorimetry trace substantially as shown in FIG. 5.

The invention further provides a composition wherein at least about 50% by weight of total 9-(S)-erythromycylamine in the composition is present as Form F. The present invention further provides a composition wherein at least about 90% by weight of total 9-(S)-erythromycylamine in the composition is present as Form F. The present invention further provides a composition wherein at least about 95% by weight of total 9-(S)-erythromycylamine in the composition is present as Form F.

The invention further provides a method of preparing Form F comprising dissolving and/or suspending solid 9-(S)-erythromycylamine in a solution containing, for example, ethanol or acetone/hexanes or mixtures thereof for a time and under conditions suitable for forming Form F. The invention further provides a method of preparation of Form F comprising inducing precipitation of Form F from a solution containing ethanol or acetone/hexanes or mixtures thereof by cooling or evaporation of the solution. The invention further provides a method of preparation of Form F by inducing precipitation of Form F from a solution containing ethanol or acetone/hexanes or mixtures thereof by adding isolates of substantially pure Form F to the solution.

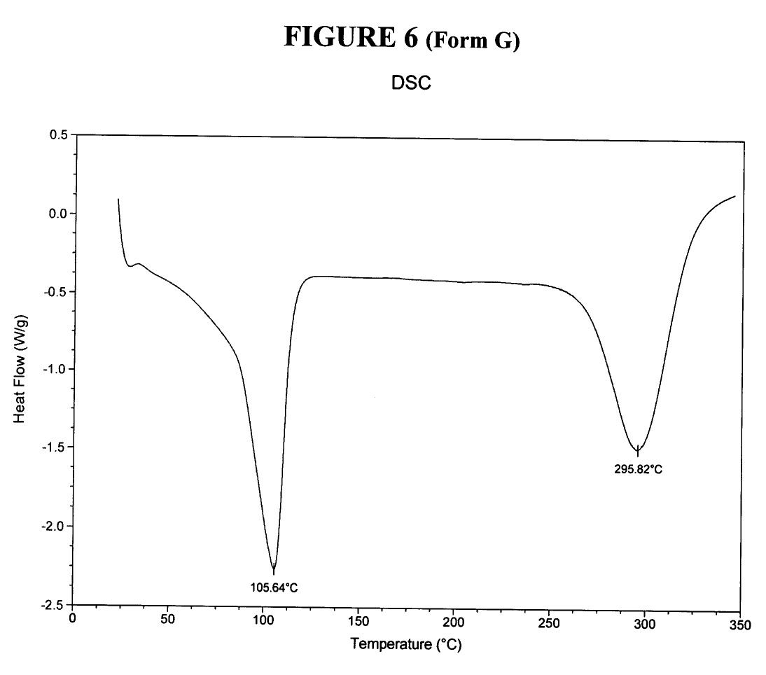

The invention further provides a crystalline form of 9-(S)-erythromycylamine designated From G. In some embodiments, Form G has a differential scanning calorimetry trace showing an endotherm between about 100 and about 110° C. In further embodiments, Form G has a differential scanning calorimetry trace substantially as shown in FIG. 6.

The invention further provides a composition wherein at least about 50% by weight of total 9-(S)-erythromycylamine in the composition is present as Form G. The invention further provides a composition wherein at least about 90% by weight of total 9-(S)-erythromycylamine in the composition is present as Form G. The present invention further provides a composition wherein at least about 95% by weight of total 9-(S)-erythromycylamine in the composition is present as Form G.

The invention further provides a method of preparing Form G comprising dissolving and/or suspending solid 9-(S)-erythromycylamine in a solution containing, for example, acetonitrile under conditions suitable for forming Form G. The invention further provides a method of preparing Form G by inducing precipitation of Form G from a solution containing, for example, acetonitrile by evaporation of the solution. The invention further provides a method of preparing Form G comprising by precipitation of Form G from a solution of containing, for example, acetonitrile by adding isolates of substantially pure Form G to the solution.

The invention further provides Form G having a powder X-ray diffraction pattern with a prominent peak, in terms of 2θ, at about 9.3°.

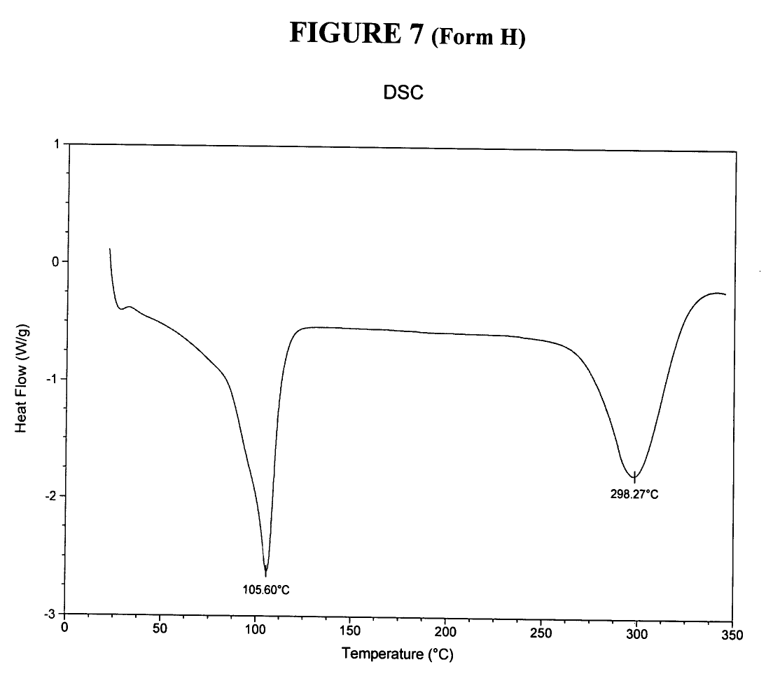

The invention further provides a crystalline form of 9-(S)-erythromycylamine designated Form H. In some embodiments, Form H has a differential scanning calorimetry trace showing an endotherm between about 100 and about 110° C. In further embodiments, Form H has a differential scanning calorimetry trace substantially as shown in FIG. 7.

The invention further provides a composition wherein at least about 50% by weight of total 9-(S)-erythromycylamine in the composition is present as Form H. The invention further provides a composition wherein at least about 90% by weight of total 9-(S)-erythromycylamine in the composition is present as Form H. The invention further provides a composition wherein at least about 95% by weight of total 9-(S)-erythromycylamine in the composition is present as Form H.

The present invention further provides a method of preparing Form H by dissolving and/or suspending solid 9-(S)-erythromycylamine in a suitable solvent such as, for example, acetonitrile under conditions suitable for forming Form H. The invention further provides a method of preparing form H by inducing precipitation of form H from a solution containing, for example, acetonitrile by cooling the solution. The invention further provides a method of preparing Form H by inducing precipitation of Form H from a solution containing, for example, acetonitrile by adding isolates of substantially pure Form H to the solution.

The invention further provides Form H having a powder X-ray diffraction pattern with a prominent peak, in terms of 2θ, at about 9.5°.

The invention further provides a crystalline form of 9-(S)-erythromycylamine designated Form G. Form I can be characterized as having a powder X-ray diffraction pattern comprising characteristic peaks, in terms of 2θ, at about 8.5° and about 10.6°. In some embodiments, Form I has a powder X-ray diffraction pattern comprising at least 5 characteristic peaks, in terms of 2θ, selected from the group consisting of about 8.5°, about 10.6°, about 10.9°, about 17.8°, about 19.4°, and about 21.8°.

The invention further provides a composition wherein at least about 50% by weight of total 9-(S)-erythromycylamine in the composition is present as Form I. Further embodiments provide a composition wherein at least about 90% by weight of total 9-(S)-erythromycylamine in the composition is present as Form I. Further embodiments provide a composition wherein at least about 95% by weight of total 9-(S)-erythromycylamine in the composition is present as Form I.

The invention further provides a method of preparing Form I by dissolving and/or suspending solid 9-(S)-erythromycylamine in a suitable solvent such as, for example, methanol/acetonitrile under conditions suitable for forming Form I. The invention further provides a method of preparing Form I by inducing precipitation (e.g., crash precipitation) of Form I from a solution containing, for example, a suitable solvent such as ethanol or methanol/acetonitrile. Further embodiments provide a method of preparing Form I by inducing precipitation of Form I from a solution containing, for example, methanol/acetonitrile by adding isolates of substantially pure Form I to the solution.

The invention further provides a pharmaceutical composition comprising a combination of Form A and Form I of 9-(S)-erythromycylamine.

The invention further provides a crystalline form (Form J) of 9-(S)-erythromycylamine. Form J can be characterized as having a powder X-ray diffraction pattern comprising characteristic peaks, in terms of 2θ, at about 17.1° and about 21.8°. In some embodiments, Form J has a powder X-ray diffraction pattern comprising at least 3 characteristic peaks, in terms of 2θ, at about 17.1°, about 19.5°, about 21.8°, about 22.6°, about 23.1° and about 23.5°.

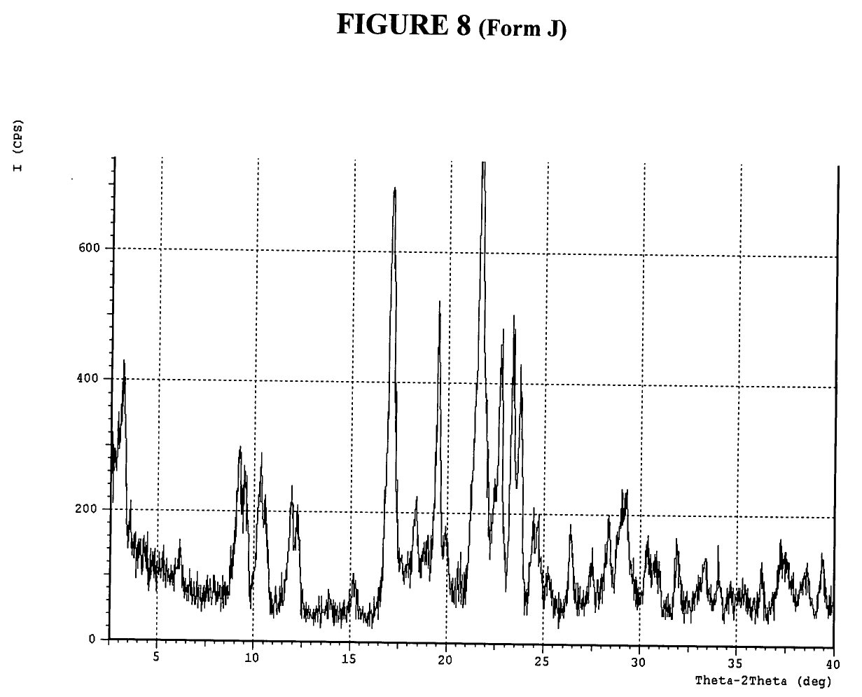

The invention further provides Form J having a powder X-ray diffraction pattern substantially as shown in FIG. 8.

The invention further provides a composition wherein at least about 50% by weight of total 9-(S)-erythromycylamine in the composition is present as Form J. Further embodiments provide a composition wherein at least about 90% by weight of total 9-(S)-erythromycylamine in the composition is present as Form J. Further embodiments provide a composition wherein at least about 95% by weight of total 9-(S)-erythromycylamine in the composition is present as Form J.

The invention further provides a method of preparing Form J by dissolving and/or suspending solid 9-(S)-erythromycylamine in a suitable solvent such as, for example, water/isopropanol under conditions suitable for forming Form J. The invention further provides a method of preparing Form J wherein solid 9-(S)-erythromycylamine in a solution containing water/isopropanol is heated to about 40° C. The invention further provides a method of preparing Form J by inducing precipitation of Form J from a solution of water/isopropanol by cooling or evaporation of the solution. The invention further provides a method of preparing Form J by inducing precipitation of Form J from a solution of ethanol or water/isopropanol by adding isolates of substantially pure Form J to the solution.

The invention further provides a composition comprising at least two crystalline forms of 9-(S)-erythromycylamine selected from the group consisting of Form A, Form B, Form C, Form D, Form E, Form F, Form G, Form H, Form I, and Form J.

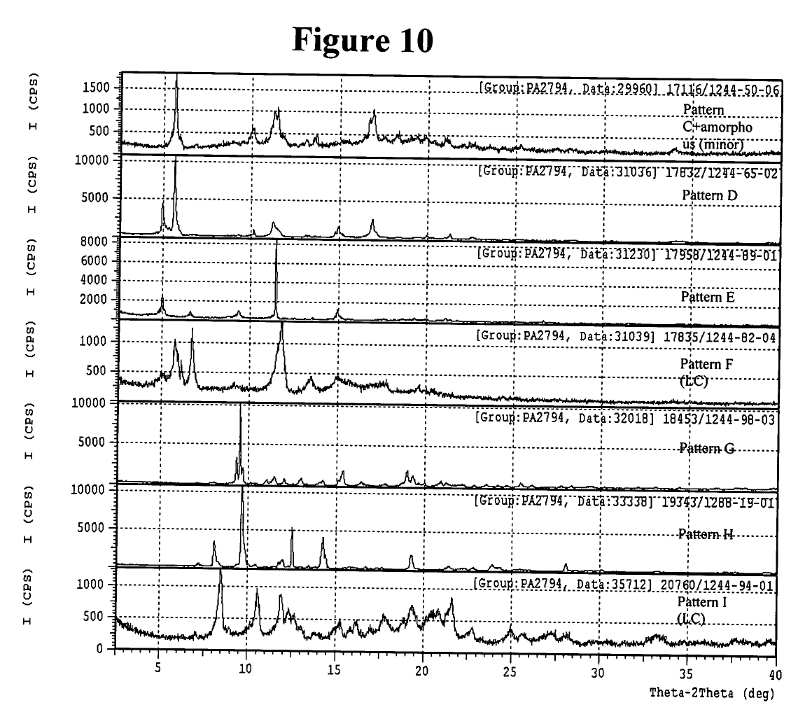

The invention further provides Form C having a having a powder X-ray diffraction pattern substantially as shown in FIG. 10.

The invention further provides Form D having a having a powder X-ray diffraction pattern substantially as shown in FIG. 10.

The invention further provides Form E having a having a powder X-ray diffraction pattern substantially as shown in FIG. 10.

The invention further provides Form F having a having a powder X-ray diffraction pattern substantially as shown in FIG. 10.

The invention further provides Form G having a having a powder X-ray diffraction pattern substantially as shown in FIG. 10.

The invention further provides Form H having a having a powder X-ray diffraction pattern substantially as shown in FIG. 10.

The invention further provides Form I having a having a powder X-ray diffraction pattern substantially as shown in FIG. 10.

The invention further provides a method of treating a bacterial or protozoal infection in a patient comprising administering to the patient a therapeutically effective amount of a crystalline form of 9-(S)-erythromycylamine selected from Form A, Form B, Form C, Form D, Form E, Form F, Form G, Form H, Form I, and Form J. The invention further provides a method of treating a bacterial or protozoal infection in a patient comprising administering to the patient a therapeutically effective amount of a composition containing 9-(S)-erythromycylamine present as Form A, Form B, Form C, Form D, Form E, Form F, Form G, Form H, Form I, or Form J. In further embodiments, the bacterial or protozoal infection is a respiratory infection. In a more particular embodiment, the bacterial or protozoal infection is a respiratory infection. In a more particular embodiment, the respiratory infection is severe chronic bronchitis.

The invention further provides a pharmaceutical composition containing Form A, Form B, Form C, Form D, Form E, Form F, Form G, Form H, Form I, or Form J of 9-(S)-erythromycylamine. The crystalline forms of the invention have antibacterial activity and can be used in methods of treatment (including prophylaxis) of a bacterial or protozoal infection in an individual (e.g., patient) by administering to the individual a therapeutically effective amount or dose of a crystalline form of the invention or a pharmaceutical composition thereof. The bacterial or protozoal infection can localized or systemic. In some embodiments, the bacterial or protozoal infection can be a respiratory infection such as severe chronic bronchitis.

As used herein the terms “bacterial infection” and “protozoal infection” include bacterial infections and protozoal infections that occur in mammals, fish and birds as well as disorders related to bacterial infections and protozoal infections that may be treated or prevented by antibiotics such as the crystalline forms of the invention. Such bacterial infections and protozoal infections, and disorders related to such infections, include the following: pneumonia, otitis media, sinusitis, bronchitis (including severe chronic bronchitis), tonsillitis and mastoiditis, each of which can be related to infection by Staphylococcus pneumoniae, Haemophilus influenzae, Moraxella catarrhalis, Staphlococcus aureus, or Peptostreptococcus spp.; pharynigis, rheumatic fever and glomerulonephritis related to infection by, for example, Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by, for example, Staphlococcus aureus, coagulase-positive staphlococci (i.e., S. epidermis., S. hemolyticus, etc.), Staphylococcus pyogenes, Streptococcus agalactiae, Streptococcal groups C-F (minute-colony streptococci), Viridans streptococci, Corynebacterium minutissimum, Clostridium spp., or Bartonella henselae; uncomplicated acute urinary tract infections related to infection by, for example, Staphylococcus saprophyticus or Enterococcus spp.; urethritis and cervicitis; and sexually transmitted diseases related to infection by, for example, Chlamydia trachomatis, Haemophilus ducreyi, Treponema pallidum, Ureaplasma urealyticum, or Neiserria gonorrhea; toxin diseases related to infection by S. aureus (food poisoning and Toxic Shock Syndrome), or Groups A, B and C streptococci; ulcers related to infection by Helicobacterpylori, systemic febrile syndromes related to infection by, for example, Borrelia recurrentis; Lyme disease related to infection by Borrelia burgdorferi, conjunctivitis, keratitis, and dacrocystitis related to infection by, for example, Chlamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni, intestinal protozoa related to infection by Cryptosporidium spp.; odontogenic infection related to infection by Viridans streptococci; persistent cough related to infection by Bordetella pertussis; gas gangrene related to infection by Clostridium perfringens or Bacteroides spp.; and atherosclerosis related to infection by Helicobacter pylori or Chlamydia pneumoniae.

Bacterial infections and protozoal infections and disorders related to such infections that can be treated or prevented in animals include the following: bovine respiratory disease related to infection by P. haem., P. multocida, Mycoplasma bovis, or Bordetella spp.; cow enteric disease related to infection by E coli or protozoa (i.e., coccidia, cryptosporidia, etc.); dairy cow mastitis related to infection by Staph. aureus, Strep. uberis, Strep. agalactiae, Strep. dysgalactiae, Klebsiella spp., Corynebacterium, or Enterococcus spp.; swine respiratory disease related to infection by A. pleuro., P. multocida or Mycoplasma spp.; swine enteric disease related to infection by E coli Lawsonia intracellularis, Salmonella, or Serpulina hyodyisinteriae; cow footrot related to infection by Fusobacterium spp.; cow metritis related to infection by E coli; cow hairy warts related to infection by Fusobacterium necrophorum or Bacteroides nodosus; cow pink-eye related to infection by Moraxella bovis; cow premature abortion related to infection by protozoa (i.e., neosporium) urinary tract infection in dogs and cats related to infection by E coli, skin and soft tissue infections in dogs and cats related to infection by Staph. epidermidis, Staph. intermedius, coagulase neg. Staph. or P. multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella. Other bacterial infections and protozoal infections and disorders related to such infections that may be treated or prevented in accord with the methods of the invention are referred to in Sanford, J. P., et aL, “The Sanford Guide To Antimicrobial Therapy,” 27 th Edition (Antimicrobial Therapy, Inc., 1996).

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting further development of the pathology and/or symptomatology); and
(3) ameliorating the disease; for example, ameliorating a disease, condition or disorder in an individual that is experiencing or displaying the pathology or symptomatology of the disease, condition or disorder (i.e., reversing the pathology and/or symptomatology).

When employed as a pharmaceutical, the crystalline form of the invention can be administered in the form of a pharmaceutical composition. The composition can be administered by a variety of routes including oral, rectal, transdermal, subcutaneous, intravenous, intramuscular, and intranasal, and can be prepared in a manner well known in the pharmaceutical art.

This invention also includes pharmaceutical compositions which contain, as the active ingredient, the crystalline form of the invention in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

While the compounds of the invention can be administered as the sole active pharmaceutical agent, they can also be used in combination with one or more other agents used in the treatment of disorders. Representative agents useful in combination with the compounds of the invention for the treatment of bacterial and/or protozoal infections include, for example, antibiotics.

When additional active agents are used in combination with a crystalline form of 9-(S)-erythromycylamine of the present invention, the additional active agents may generally be employed in therapeutic amounts as indicated in the PHYSICIANS' DESK REFERENCE (PDR) 53^{rd} Edition (1999), which is incorporated herein by reference, or such therapeutically useful amounts as would be known to one of ordinary skill in the art.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner. In some embodiment, the composition contains a dry powder where the mass medium diameter is predominately between 1 to 5 microns for effective administration to the lungs such as the lung endobronchial space, including alveoli.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgement of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the drug, formulation of the excipient, and its route of administration. Effective doses can be extrapolated from dose-response curves derived from in vitro or animal model test systems.

In order that the invention disclosed herein may be more efficiently understood, examples are provided below. It should be understood that these examples are for illustrative purposes only and are not to be construed as limiting the invention in any manner.

### EXAMPLES

### Example 1

### Solid State Preparation of Form A of 9-(S)-Erythromycylamine

Preparation A

Four different commercial samples of 9-(S)-erythromycylamine were placed in an oven in open glass containers and gradually heated to about 150° C. over the course of 15 minutes (49-53 mg) or gradually heated to about 134° C. over the course of 1 hour (about 100 mg). Starting material characteristics, determined by DSC and XRPD substantially as described in Examples 2 and 3, are provided in Table II below.

TABLE II

| Sample | Prior to heating |
|---|---|
| 1 | Composed primarily of a |
| | polymorph other than Form A |
| 2 | Mixture of amorphous material and |
| | Form A |
| 3 | Composed primarily of a |
| | polymorph other than Form A |
| 4 | Primarily amorphous material |

Thermograms of the heat-treated samples were obtained by DSC substantially as described in Example 3. Heat treatment of all samples under both conditions resulted in an increase in the amount of Form A (observed in all samples as an increase in amplitude of a sharp endotherm occurring between 190 and 200° C.). Samples 1, 3 and 4, which initially contained little or no Form A, appeared to contain primarily Form A after heat treatment. For Sample 2, which contained a mixture of Form A and amorphous material, the product appeared to contain an increased amount of Form A and decreased amount of amorphous material as confirmed by XRPD.

Preparation B

Samples of solid 9-(S)-erythromycylamine are placed in an oven in open glass containers and heated to at least 170° C. for greater than 10 minutes (10-100 mg) or heated to 190° C. for over 5 minutes (about 10-100 mg). Samples will subsequently contain a greater percentage of crystalline Form A.

Preparation C

Three samples (about 100 mg each) of amorphous solid 9-(S)-erythromycylamine were placed in an oven in open glass containers. One sample was heated to 150° C. for 15 min, one sample was heated to 160° C. for 15 min, and one sample was heated to 170° C. for 15 minutes. According to XRPD analysis, the sample heated to 150° C. remained largely amorphous, while the samples heated to 160° C. and 170° C. displayed crystallinity corresponding to Form A.

### Example 2

### X-Ray Powder Diffraction

X-ray powder diffraction (XRPD) analyses were performed using a Shimadzu XRD-6000 X-ray powder diffractometer using Cu Kα radiation. The instrument was equipped with a fine focus X-ray tube. The tube voltage and amperage were set to 40 kV and 40 mA, respectively. The divergence and scattering slits were set at 1° and the receiving slit was set at 0.15 mm. Diffracted radiation was detected by a Nal scintillation detector. A theta-two theta continuous scan at 3°/min (0.4 sec/0.02° step) from 2.5 to 40° 2θ was used. A silicon standard was analyzed to check the instrument alignment. Data were collected and analyzed using XRD-6000 v. 4.1. Samples were prepared for analysis by placing them in a silicon sample holder or aluminum holder with silicon insert.

### Example 3

### Differential Scanning Calorimetry

Differential scanning calorimetry (DSC) was performed using a TA Instruments differential scanning calorimeter 2920. The sample was placed into an aluminum DSC pan, and the weight was recorded. The pan was covered with a lid and was not crimped. The sample cell was equilibrated at 25° C. and heated under a nitrogen purge at a rate of 10° C./min, up to a final temperature of 350° C. Indium metal was used as the calibration standard.

An example DSC thermogram that is consistent with Form A provided in FIG. 2. The thermogram is characterized as having a sharp endotherm at about 192° C. corresponding to a melting event. The endotherm also shows a second endotherm at about 296° C. and a slight baseline shift at about 111° C.

### Example 4

### Thermogravimetric Analysis

Thermogravimetric (TG) analyses were performed using a TA Instruments 2950 and 2050 thermogravimetric analyzer. Each sample was placed in an aluminum sample pan and inserted into the TG furnace. The sample was first equilibrated at 25° C., and then heated under nitrogen at a rate of 10° C./min, up to a final temperature of 350° C. Nickel and Alumel™ were used as the calibration standards.

TG data carried out in connection with Form A shows a 0.4% weight loss up to 190° C. which is consistent with material that is not solvated.

### Example 5

### Moisture Sorption/Desorption

Moisture sorption/desorption data were collected on a VTI SGA-100 Vapor Sorption Analyzer. Sorption and desorption data were collected over a range of 5% to 95% relative humidity (RH) at 10% RH intervals under a nitrogen purge. Samples were not dried prior to analysis. Equilibrium criteria used for analysis were less than 0.0100% weight change in 5 minutes, with a maximum equilibration time of 3 hours if the weight criterion was not met. Data were not corrected for the initial moisture content of the samples. NaCl and PVP were used as calibration standards.

Samples of Form A showed minimal weight loss upon equilibration at 5% RH, and gained 2.2 % of its weight upon equilibration at 95% RH. Most of the gained weight was lost upon re-equilibration at 5% RH. Accordingly, Form A was determined to be slightly hygroscopic.

### Example 6

### Preparation/Characterization of Form B

Form B was prepared from slurries of 9-(S)-erythromycylamine in aqueous and mixed organic/aqueous solvents at various temperatures.

A thermographic curve showed a broad endothermic event at ˜66° C., likely due to a desolvation event, followed by a sharp endotherm at 116° C. (See FIG. 9). There was also a very small endotherm at 195° C., as shown in FIG. 9. A modulated DSC experiment on this lot showed that the event in the DSC curve at ˜116° C. was due to a glass transition, suggesting that upon desolvation the sample becomes amorphous. A TG-IR experiment was performed on this lot. The experiment showed that the sample loses 0.8% of its weight up to 150° C., and that the solvent present is water. The resulting material after the TG-IR experiment was analyzed by XRPD, and was found to be amorphous, which is supported by the thermal data. An XRPD spectrum of Form B is provided in FIG. 11.

Based on the characterization data, Form B is likely a hydrate that dehydrates to form amorphous material.

### Example 7

### Preparation/Characterization of Form C

Form C was prepared from evaporations of 9-(S)-erythromycylamine in ethanol or isopropanol, from slurries of 9-(S)-erythromycylamine in ethanol, or from antisolvent crystallizations from acetonitrile/isopropanol and methanol/isopropanol. A representative XRPD pattern is shown in FIG. 10. The sample showed a 1.1% weight loss up to 200° C. The DSC showed a broad endotherm at 74° C., followed by a small endotherm at 113° C., and a very small, broad exotherm at 163° C. Hotstage microscopy showed that the event at ˜104° C. corresponds to a decrease in birefringence of the sample and a softening of the sample. The solids start to melt above 123° C. A TG-IR experiment on a sample of Form C prepared from slurrying amorphous material in ethanol for 8 days showed a larger weight loss (6.9% up to 120° C.), and the experiment showed that the solvent present was ethanol. This material appears to be weakly solvated since the solvent loss starts at ambient temperature. The XRPD pattern of the material after the TG-IR experiment showed that it was amorphous, indicating that upon desolvation the sample became amorphous. Based on the characterization data, Form C is a crystalline solvated material that can likely exist as a series of iso-structural solvates.

### Example 8

### Preparation of Form D

Form D was prepared from isopropanol solutions of 9-(S)-erythromycylamine, or from antisolvent crystallizations using dichloromethane/isopropanol or toluene/isopropanol. A representative XRPD pattern is shown in FIG. 10. The sample showed a weight loss of 3.9% up to 200° C., suggesting that the material is solvated. The DSC of FIG. 4 showed a broad endotherm at 80° C., followed by two sharper endotherms at 105 and 110° C. and a small, broad exotherm at 185° C. Hotstage microscopy showed a loss of birefringence at ˜110° C., and the solids started to melt at ˜123° C., similar to the behavior of Form C described above. Based on the characterization data, Form D is a crystalline material that is likely an isopropanol solvate.

### Example 9

### Preparation of Form E

Form E was prepared from the evaporation of an isopropanol solution of 9-(S)-erythromycylamine, or from antisolvent crystallizations using acetonitrile/isopropanol or methanol/isopropanol. A representative XRPD pattern is shown in FIG. 10. The sample showed a weight loss of 4.5% up to 200° C., suggesting that the material is solvated. The DSC in FIG. 5 showed a broad endotherm at 67° C., followed by another endotherm at 105° C. and smaller events at approximately 140, 158, and 203° C. A TG-IR experiment on a sample of Form E material prepared from the fast evaporation from anhydrous isopropanol showed a larger weight loss (6.9% up to 140° C.), and the solvent present was isopropanol. The XRPD pattern of the material after the TG-IR experiment showed that it was amorphous, indicating that upon desolvation the sample became amorphous. Based on the characterization data, the Form E is a crystalline material that is likely an isopropanol solvate.

### Example 10

### Preparation of Form F

Pattern F material was prepared from the evaporation of an ethanol solution of 9-(S)-erythromycylamine, or from antisolvent crystallizations using acetone/hexanes. The sample showed a weight loss of 1.9% up to 200° C., suggesting that the material may be solvated.

### Example 11

### Preparation of Form G

Form G was initially prepared from the evaporation of acetonitrile solutions of 9-(S)-erythromycylamine. A representative XRPD pattern is shown in FIG. 10. The DSC curve shown in FIG. 6 shows a large, broad endotherm at 106° C., followed by a broader endotherm centered at 296° C., which is likely due to decomposition, Form G was also prepared from slurries of 9-(S)-erythromycylamine in aqueous and mixed organic/aqueous solvents at several temperatures. A TG-IR experiment performed on Form G showed that the material is a hydrate. The sample shows a weight loss of 8.2% up to 70° C. suggesting the sample may contain multiple waters of hydration. The sample shows an 8.4% weight loss upon equilibration at 5% RH, and gains 7.8% of its weight upon equilibration at 95% RH. Most of this weight is lost again upon re-equilibration at 5% RH. Based on the characterization data, Form G is a crystalline hydrate that is weakly hydrated.

### Example 12

### Preparation of Form H

Pattern H material was prepared from the slow cooling of an acetonitrile solution of 9-(S)-erythromycylamine. A representative XRPD pattern is shown in FIG. 10. The thermal data for Pattern H material is shown in FIG. 7. The DSC curve shows a large, broad endotherm at 106° C., followed by a broader endotherm centered at 298° C. that is likely due to decomposition.

### Example 13

### Preparation of Form I

Pattern I material was prepared from a crash precipitation crystallization of 9-(S)-erythromycylamine using methanol/acetonitrile. A representative XRPD pattern is shown in FIG. 10. The XRPD pattern is similar, but not identical, to that of Form A, suggesting it is either a new form or a mixture of two forms.

### Example 14

### Preparation of Form J

Pattern J material was prepared from a slurry of 9-(S)-erythromycylamine in water/isopropanol at 40° C. A representative XRPD pattern is shown in FIG. 8.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A crystalline form of 9-(S)-erythromycylamine having a powder X-ray diffraction pattern comprising characteristic peaks, in terms of 2θ, at about 17.7° and about 19.1° (Form A).

2. The crystalline form of claim 1 wherein the powder X-ray diffraction pattern comprises at least 5 characteristic peaks, in terms of 2θ, selected from about 8.8°, about 10.7°, about 11.4°, about 12.9°, about 14.1°, about 15.6°, about 16.2°, about 16.6°, about 17.7, about 19.1°, about 20.3°, about 21.0°, about 21.9°, about 22.4°, about 24.0°, about 24.5°, about 24.8°, and about 26.0°.

3. The crystalline form of claim 1 having a powder X-ray diffraction pattern substantially as shown in FIG. 1.

4. The crystalline form of claim 1 having a differential scanning calorimetry trace showing an endotherm between about 190 and 200° C.

5. The crystalline form of claim 1 having a differential scanning calorimetry trace substantially as shown in FIG. 2.

6. A composition comprising the crystal form of claim 1.

7. The composition of claim 6 wherein at least about 50% by weight of total 9-(S)-erythromycylamine in said composition is present as said crystal form.

8. The composition of claim 6 wherein at least about 70% by weight of total 9-(S)-erythromycylamine in said composition is present as said crystal form.

9. The composition of claim 6 wherein at least about 80% by weight of total 9-(S)-erythromycylamine in said composition is present as said crystal form.

10. The composition of claim 6 wherein at least about 90% by weight of total 9-(S)-erythromycylamine in said composition is present as said crystal form.

11. The composition of claim 6 wherein at least about 95% by weight of total 9-(S)-erythromycylamine in said composition is present as said crystal form.

12. The composition of claim 6 wherein at least about 97% by weight of total 9-(S)-erythromycylamine in said composition is present as said crystal form.

13. The composition of claim 6 wherein at least about 98% by weight of total 9-(S)-erythromycylamine in said composition is present as said crystal form.

14. The composition of claim 6 wherein at least about 99% by weight of total erythromycylamine in said composition is present as said crystal form.

15. The composition of claim 6 further comprising a pharmaceutically acceptable carrier.

16. The composition of claim 15 that is suitable for inhalation.

17. A composition consisting essentially of 9-(S)-erythromycylamine wherein at least 95% by weight of said 9-(S)-erythromycylamine is present in said composition as said crystal form of claim 1.

18. The composition of claim 17 wherein at least 97% by weight of said 9-(S)-erythromycylamine is present in said composition as said crystal form of claim 1.

19. The composition of claim 17 wherein at least 98% by weight of said 9-(S)-erythromycylamine is present in said composition as said crystal form of claim 1.

20. The composition of claim 17 wherein at least 99% by weight of said 9-(S)-erythromycylamine is present in said composition as the crystalline form of claim 1.

21. A method of preparing the crystal form of claim 1 comprising heating solid 9-(S)-erythromycylamine for a time and under conditions suitable for forming said crystal form.

22. The method of claim 21 wherein said solid 9-(S)-erythromycylamine comprises amorphous 9-(S)-erythromycylamine.

23. The method of claim 21 wherein said solid 9-(S)-erythromycylamine is heated to about 100 to about 200° C.

24. The method of claim 21 wherein said solid 9-(S)-erythromycylamine is heated to about 170° C. for about 10 to about 30 minutes.

25. The method of claim 21 wherein said solid 9-(S)-erythromycylamine is heated to about 170 to about 190° C. for about 5 minutes to about 2 hours.

26. A method of preparing the crystal form of claim 1 comprising heating solid 9-(S)-erythromycylamine for a time and under conditions suitable for forming Form A wherein said solid 9-(S)-erythromycylamine comprises a 9-(S)-erythromycylamine crystalline form other than Form A.

27. The method of claim 26 wherein said solid 9-(S)-erythromycylamine is heated to about 100 to about 200° C.

28. The method of claim 26 wherein said solid 9-(S)-erythromycylamine is heated to at least about 160° C. for about 10 to about 30 minutes.

29. The method of claim 26 wherein said solid 9-(S)-erythromycylamine is heated to at least about 170° C. for about 10 to about 30 minutes.

30. The method of claim 26 wherein said solid 9-(S)-erythromycylamine is heated to a temperature of about 160 to about 200° C. for about 5 minutes to about 2 hours.

31. The method of claim 26 wherein said solid 9-(S)-erythromycylamine is heated to a temperature of about 170 to about 190° C. for about 5 minutes to about 2 hours.

32. A method of preparing said crystal form of claim 1 comprising inducing precipitation of said crystal form from a high boiling solvent at elevated temperature by addition of an anti-solvent.

33. The method of claim 32 wherein the elevated temperature is above about 80° C. and less than about the melting point of said crystalline form.

34. The method of claim 32 wherein the elevated temperature is about 80 to about 180° C.

35. A method of preparing the crystal form of claim 1 comprising inducing precipitation of said crystal form from a high boiling solvent at elevated temperature by evaporation of said high boiling solvent.

36. A method of preparing the crystal form of claim 1 comprising inducing precipitation of Form A from a high boiling solvent at elevated temperature by cooling of said high boiling solvent.

37. A method of treating a bacterial or protozoal infection in a patient comprising administering to said patient a therapeutically effective amount of 9-(S)-erythromycylamine comprising the crystalline form of claim 1.

38. The method of claim 37 wherein the bacterial or protozoal infection is a respiratory infection such as severe chronic bronchitis.
